(19) United States
(12) Patent Application Publication
(10) Pub. No.: US 2025/0164496 A1
(43) Pub. Date: May 22, 2025

(54) SINGLE CELL PROTEOMICS USING DEGRADABLE HYDROGELS

(71) Applicant: Cellanome, Inc., Foster City, CA (US)

(72) Inventors: Pier Federico GHERARDINI, Redwood City, CA (US); Tarun Kumar KHURANA, Fremont, CA (US); Stephen C. Macevicz, Bozeman, MT (US)

(21) Appl. No.: 19/030,206

(22) Filed: Jan. 17, 2025

Related U.S. Application Data
(63) Continuation of application No. PCT/US2023/070428, filed on Jul. 18, 2023.
(60) Provisional application No. 63/390,512, filed on Jul. 19, 2022.

(51) Int. Cl. G01N 33/68; G01N 33/543

(52) U.S. Cl. CPC G01N 33/6803; G01N 33/543

(57) ABSTRACT

The present disclosure is directed to methods for measuring proteomes of single cells. In one aspect, methods comprise disposing cells on a surface, synthesizing hydrogel chambers around each of the cells, lysing the cells to release intracellular proteins for adsorption onto the surface bounded by the hydrogel chambers, depolymerizing the hydrogel chambers, and quantifying the adsorbed proteins with detection antibodies to determine the single cell proteomes.

### CROSS REFERENCE

This application is a continuation of International Application No. PCT/US2023/070428, filed Jul. 18, 2023, which claims the benefit of U.S. Provisional Application No. 63/390,512 filed Jul. 19, 2022, which applications are incorporated herein by reference in their entirety for all purposes.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

A hallmark of biology is degeneracy, Edelman et al, Proc. Natl. Acad. Sci., 98(24): 13763-13768 (2001). Degeneracy is the ability of elements, such as cells, that are structurally different to perform the same function or yield the same output, and to perform a different function or yield a different output depending on the context in which it is expressed. Examples of degenerate systems are widespread in biology, and include development, immune responses, cancer and functional neuro-anatomy. To understand the operation of such degenerate systems it is necessary to be able to characterize and measure properties of individual cells, such as, their transcriptomes, proteomes, metabolomes, and the like. Techniques that provide only cellular averages of such properties are unable to reveal the cellular heterogeneity necessary for a full understanding of such biological processes, e.g. Dittrich et al, Anal. Bioanal. Chem., 406: 6957-6961 (2014); Lindstrom et al, editors, Single-Cell Analysis: Methods and Protocols (Humana Press, 2012). Over the last decade, striking progress has been made in transcriptome analysis of single cells and tissues, e.g. Saliba et al, Nucleic Acids Research, 42(14); 8845-8860) (2014): Wang et al, Molecular Cell, 58: 598-609 (2015); Lee et al, Experimental & Molecular Medicine, 52: 1428-1442 (2020); and the like. However, comparable progress has not occurred in proteome analysis, Vistain et al, Trends Biochemical Sciences, 46(8): 661-672 (2021).

In view of the above, the availability of new methods and apparatus for efficient and convenient analysis of single cell proteomes would advance our understanding of complex degenerate systems in biology.

### SUMMARY

The present disclosure is directed to a method of determining proteomes of single cells comprising: (a) synthesizing one or more gel chambers enclosing each of the one or more cells disposed on a surface of a channel; (b) lysing the cells so that cellular proteins of each cell are released into its hydrogel chamber and at least a portion are adsorbed onto the surface enclosed by the gel chamber; and (c) detecting the adsorbed proteins with detection antibodies. In some embodiments, the method further comprises collecting optical signals from the one or more cells disposed on the surface; and determining, prior to synthesizing, the position of each of the one or more cells from the optical signals. In some embodiments, the method further comprises depolymerizing the hydrogel chambers prior to detecting. In some embodiments, the method further comprises de-adsorbing the adsorbed proteins prior to depolymerizing.

In some embodiments, the method of determining proteomes of single cells comprises (a) providing a fluidics device comprising: (i) a channel comprising a surface, (ii) a spatial energy modulation element in optical communication with the surface, and (iii) a detector in optical communication with the surface and in operable association with the spatial energy modulating element, the detector collecting one or more optical signals for identifying cells and determining positions thereof on the surface; (b) loading the channel with cells and one or more polymer precursors so that the cells are disposed on or adjacent to the surface; (c) synthesizing one or more hydrogel chambers enclosing each of the one or more cells by projecting light into the channel with the spatial energy modulating element such that the projected light causes cross-linking of the one or more polymer precursors to form hydrogel chambers, wherein the positions of the hydrogel chambers in the channel are determined by the positions of the cells enclosed thereby identified by the detector; (d) lysing the cells so that [at least a portion of] cellular proteins of each cell are released into its hydrogel chamber and are adsorbed onto the surface [enclosed by the hydrogel chamber]; (e) depolymerizing the hydrogel chambers; and (f) loading detection antibodies for identifying the adsorbed proteins.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure.

### BRIEF DESCRIPTION OF THE DRAWINGS

The novel features of the present disclosure are set forth with particularity in the appended claims. A better understanding of the features and advantages described herein will be obtained by reference to the following detailed description that sets forth illustrative embodiments, in which the principles of the systems and methods described herein are utilized, and the accompanying drawings (also “Figure” and “FIG.” herein), of which:

FIGS. 1A-1C illustrate embodiments for analyzing the proteomes of single cells.

FIGS. 2A-2C illustrate an embodiment for linking an antibody barcode of a bound antibody with an adjacent spatial barcode.

FIG. 2D illustrates an embodiment in which antibody barcodes of two bound antibodies are separately linked to adjacent spatial barcodes, wherein a readout of the barcodes provides a proximity assay of an adsorbed protein.

FIG. 2E illustrates the embodiment of FIGS. 2A-2C wherein released proteins are captured by antibodies attached to a surface.

FIGS. 3A-3B illustrate an embodiment wherein proteins are adsorbed onto bead surfaces having different physical and/or chemical characters.

FIGS. 4A-4B illustrate in greater detail instruments for detecting cells and synthesizing hydrogel chambers.

FIGS. 5A-5B illustrate a flow cell with multiple channels for use with some embodiments described herein.

### DETAILED DESCRIPTION

The practice of the present disclosure may employ, unless otherwise indicated, conventional techniques and descriptions of organic chemistry, molecular biology (including recombinant techniques), cell biology, and biochemistry, which are within the skill of the art. Such conventional techniques include, but are not limited to, preparation of synthetic polynucleotides, monoclonal antibodies, antibody display systems, cell and tissue culture techniques, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PCR Primer: A Laboratory Manual; Retroviruses; and Molecular Cloning: A Laboratory Manual (all from Cold Spring Harbor Laboratory Press); Renault and Duchateau, Editors, Site-directed Insertion of Transgenes (Springer, Heidelberg, 2013); Lutz and Bornscheuer, Editors, Protein Engineering Handbook (Wiley-VCH, 2009); and the like. Guidance for selecting materials and components to carry out particular functions may be found in available treatises and references on scientific instrumentation including, but not limited to, Moore et al, Building Scientific Apparatus, Third Edition (Perseus Books, Cambridge, MA); Hermanson, Bioconjugate Techniques, 3rd Edition (Academic Press, 2013); and like references.

The present disclosure is directed to methods for measuring the proteomes of single cells. As used herein, “proteome” generally means a complete set of proteins expressed by a single cell. In some embodiments, the term “proteome” encompasses a complete set of proteins expressed by a single cell including their identities and their quantities or relative quantities. In some embodiments, the term “proteome” means a defined subset of proteins expressed by a single cell. In some embodiments, such a subset may be defined by a set of antibodies used to capture and/or detect proteins of the subset. The size of such a defined subset may vary widely. In some embodiments, such a subset may comprise from 2 to 100 different proteins, or from 10 to 50 different proteins, or from 10 to 20 different proteins. The term “antibody” is intended to encompass any specific binding compound; that is, a molecule (usually a macromolecule), that specifically binds to a given protein. In particular, the term “antibody” is intended to encompass antibody fragments, aptamers, and like compounds. By “specific binding” in reference to a specific binding compound (such as an antibody) means that the specific binding compound binds (under physiological conditions) solely to its intended target protein with little or no cross-binding to (i.e. cross-reaction with) other proteins of a designated subset.

In one aspect, methods described herein comprise (a) synthesizing a gel enclosure, or chamber, around each of a plurality of cells disposed on a surface of a channel, (b) lysing the enclosed cells to release proteins which are adsorbed onto the portion of the channel surface enclosed by the gel chamber, (c) depolymerizing the gel chambers, and (d) detecting with detection antibodies the adsorbed proteins at the locations of the depolymerized gel chambers. In some embodiments, methods described herein further comprise collecting optical signals from cells disposed on the surface, including the position of each cell on the surface, prior to synthesizing the gel enclosures. The term “gel enclosure” is used interchangeably with term “gel chamber.” The composition of such gel enclosures or gel chambers may vary widely, as discussed further below. In some embodiments, such gel enclosures or gel chambers comprise hydrogels.

In some embodiments, the present disclosure comprises fixing and permeabilizing cells. In some embodiments, cells are fixed and permeablilized after synthesizing chambers and before application of binding compounds specific for such intracellular targets. In some embodiments, fixing and permeabilizing is performed in place of lysing. Fixing and permeablization of cells may be carried out by conventional protocols, such as used in flow cytometry. Typically such protocols include a steps of treating cells with a fixing agent followed by a step of treating cells with a permeabilizing agent. A fixing step typically immobilizes intracellular cellular targets, while retaining cellular and subcellular architecture and permitting unhindered access of antibodies and/or hybridization probes to all cells and subcellular compartments. Wide ranges of fixatives are commercially available, and the correct choice of method will depend on the nature of the targets being examined and on the properties of the antibody and/or hybridization probes used. Fixation methods fall generally into two classes: organic solvents and cross-linking reagents. Organic solvents such as alcohols and acetone remove lipids and dehydrate the cells, while precipitating the proteins on the cellular architecture. Cross-linking reagents (such as paraformaldehyde) form intermolecular bridges, normally through free amino groups, thus creating a network of linked antigens. Cross-linkers preserve cell structure better than organic solvents, but may reduce the antigenicity of some cell components, and require the addition of a permeabilization step, to allow access of the antibodies and/or hybridization probes to the intracellular targets. Fixing and permeabilizing steps include, but are not limited to, methanol-acetone fixation (fix in cooled methanol. 10 minutes at −20° C.: permeabilize with cooled acetone for 1 min at −20° C.); paraformaldehyde-triton fixation (fix in 3-4% paraformaldehyde for 10-20 min; rinse with phosphate buffered saline (PBS): permeabilize with 0.5% Triton X-100 for 2-10 min): paraformaldehyde-methanol fixation (fix in 3-4% paraformaldehyde for 10-20 min; rinse with PBS; permeabilize with cooled methanol for 5-10 min at −20° C.). Permeabilizing agents include, but are not limited to, detergents saponin, Triton X-100, Tween-20, NP40. Permeabilizing agents may also include proteinases, such as proteinase K, streptolysin O, and the like.

In some embodiments, methods described herein comprise synthesizing one or more gel chambers enclosing each of the one or more cells disposed on a surface of a channel; fixing and permeabilizing the cells so that intracellular proteins of each cell are accessable to detection antibodies; and detecting the intracellular proteins with the detection antibodies. In some embodiments, such one or more cells may be nonadherent cells. In some embodiments such methods further comprise collecting optical signals from the one or more cells disposed on the surface; and determining, prior to synthesizing, the position of each of the one or more cells from the optical signals.

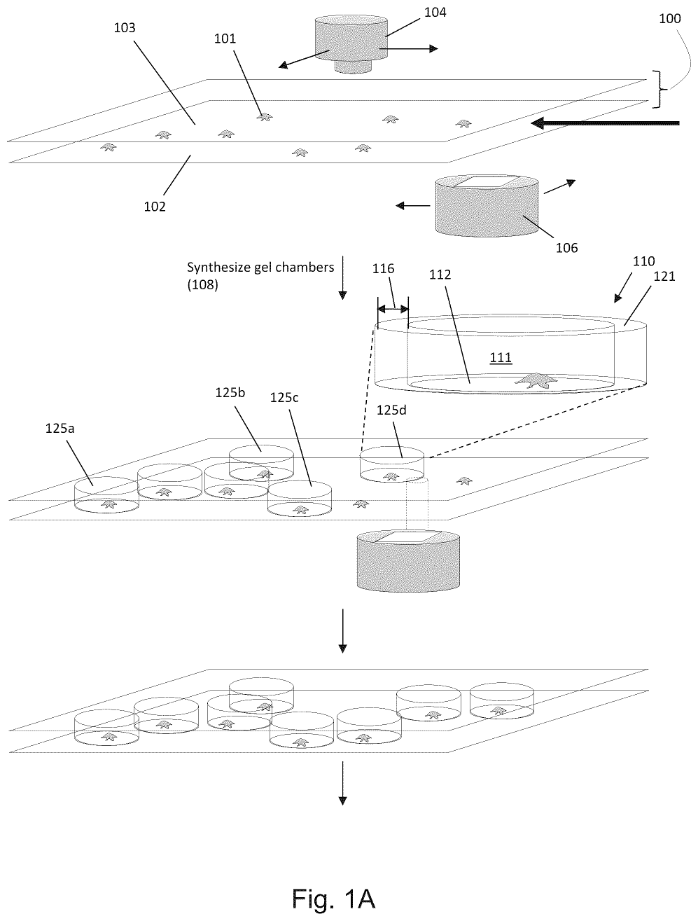

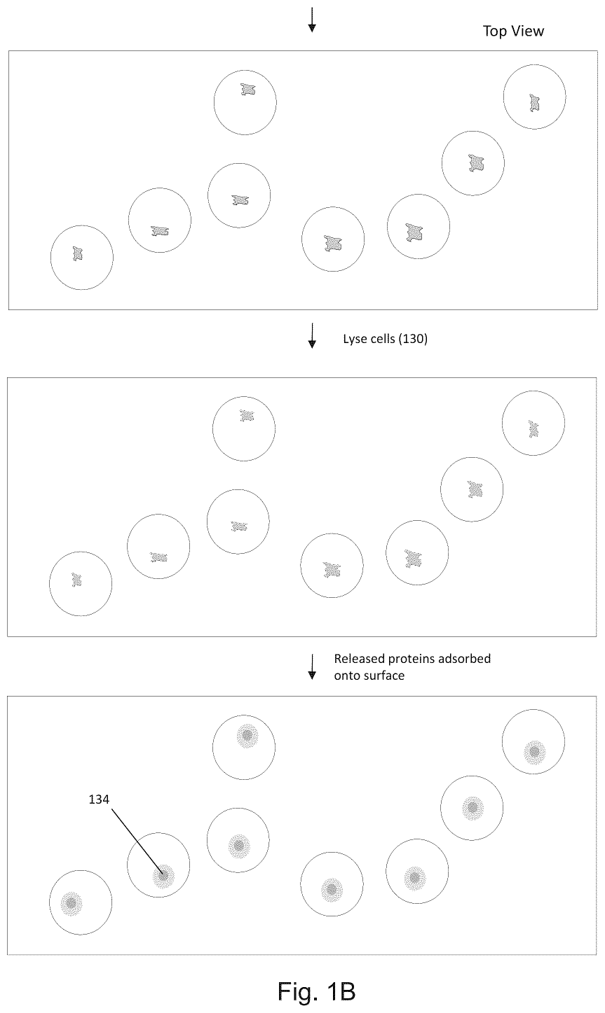

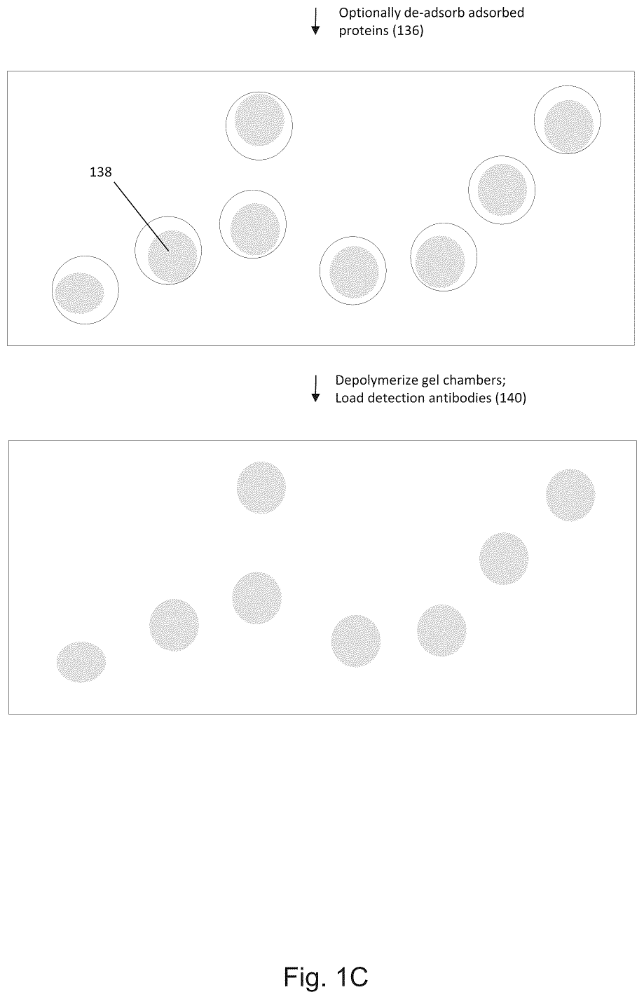

FIGS. 1A-1C illustrate one embodiment of the present disclosure. Cells (e.g. 101) are disposed on surface (102) of channel (100) formed by surfaces (102) and (103), which, for example, may be surfaces of two parallel plates of glass and/or plastic, after which cell positions are determined by detector (104). Gel enclosures, or chambers, are synthesized (108) from photo-synthesizable polymer precursors in channel (100) using light source (106). In some embodiments, detector (104) comprises a microscope and an image recognition system that is operationally associated with light source (106) to permit the positioning of gel chambers around cells (101). Detector (104) may also collect and record optical signals from cells (101) indicative of their type, state of health, function, or the like, which can be correlated with the proteomes of the cells. In some embodiments, cells may be mixed with polymer precursors (from which gels are photosynthesized) prior to being loaded together into channel (100). As described more fully below, gel chambers (e.g., 125a-d and blow-up 110) may vary widely in size and shape. For simplicity, gel chambers (125a-d and others of FIG. 1A) are illustrated as cylindrical solids, but as illustrated by blow-up (110), gel chambers may comprise walls and have non-gel interiors, as illustrated by wall (121) with thickness (116) enclosing interior space (111) with enclosed interior surface (112). In some embodiments, wall (121) extends from surface (102) to surface (103). After cells are enclosed in gel chambers, as shown in the uppermost top view of FIG. 1B, they are lysed (130). Lysing may be accomplished using conventional cell lysing techniques including, but not limited to, photo-based lysing, chemical lysing, heat-based lysing, or the like. In some embodiments, cells are lysed using a chemical lysing agent that readily passes through the walls of the gel chambers. Lysis conditions (and/or reagents) may include, but are not limited to, the following: 1) cells in H2O at 96° C. for 15 min, followed by 15 min at 10° C.; 2) 200 mM KOH, 50 mM dithiotheitol, heat to 65° C. for 10 min: 3) for 4 μL protease-based lysis buffer: 1 μL of 17 μM SDS combined with 3 μL of 125 μg/mL proteinase K, followed by incubation at 37° C. for 60 min, then 95° C. for 15 min (to inactivate the proteinase K); 4) for 10 μL of a detergent-based lysis buffer: 2 μL H2O, 2 μL 10 mM EDTA, 2 μL 250 mM dithiothreitol, 2 μL 0.5% N-laurylsarcosin salt solution; 5) 200 mM Tris pH7.5, 20 mM EDTA, 2% sarcoyl, 6% Ficoll.

As cell walls and cell membranes are broken down by a lysing agent, proteins are released into the interior of the chambers and adsorb onto interior surfaces. In some embodiments, interior surfaces of gel chambers may be selected so that protein preferentially adsorb onto a desired interior surface. For example, in some embodiments, the plate or wall comprising surface (102) may comprise a material, such as a plasma-treated plastic, that preferentially adsorbs proteins and the plate or wall comprising surface (103) may comprise a material, such as surface-passivated glass, that resists protein adsorption. Such material selections are well-understood in the field of biosensor technology, as exemplified by the following references: Lichtenberg et al, Sensors, 19: 2488 (2019); Reimhult et al, Sensors, 15: 1635-1675 (2015); Recek et al, Molecules, 18: 12441-12463 (2013); and the like. Likewise, gel polymer precursors may be selected to form gels that minimize non-specific adsorption of proteins to gel surfaces or structures. A wide variety of materials may be used for protein adsorption surfaces including, but not limited to, a non-polar surface, a hydrophobic surface, or a hydrophilic surface. In some embodiments, a surface is formed on a plastic. In some embodiments, an oxygen plasma can be used to introduce polar functional groups to a surface and make it hydrophilic. In some embodiments, a tetrafluoromethane plasma can be used to introduce non-polar functional groups to a surface and make it hydrophobic. In some embodiments, a surface may be part of a commercially available protein adsorbing material, such as MaxiSorp® material (ThermoFisher Scientific).

In some embodiments, the surface may have attached one or more capture antibodies specific for selected proteins. In some embodiments, such selected proteins may be intracellular proteins. In other embodiments, such selected proteins may be cell membrane proteins, or both intracellular proteins and cell membrane proteins. As used herein, the term “adsorption” includes the specific binding of proteins to antibodies attached to a surface. A wide variety of methods are available for immobilizing or covalently bonding antibodies to surfaces, e.g. reviewed in Trilling et al, Analyst, 138: 1619-1627 (2013); Gao et al, Analytica Chimica Acta, 1189: 338907 (2022).

In some embodiments, after released proteins are adsorbed onto the desired surface(s), walls (e.g. 134) of gel chambers are depolymerized to enhance access of detection antibodies to the adsorbed proteins. In other embodiments, after released proteins are adsorbed onto the desired surface(s), walls of gel chambers are left intact and wherein the porosity of the gel walls is selected to permit ready access of detection antibodies. Optionally, as illustrated in FIG. 1C, the distribution of adsorbed proteins may be modified by one or more steps of de-adsorption (136) that allow the de-adsorbed proteins to diffuse throughout the interior of their gel chamber to form a layer of more uniform density (138). After such modification, gel chambers may be depolymerized as described above and the adsorbed proteins exposed to detection antibodies (140). Such de-adsorption steps may be implemented by treating adsorbed proteins with heat and/or de-adsorption agents, such as, a non-ionic detergent, like for example, Brij, Triton, Tween, or the like. In some embodiments, de-adsorption may be implemented by increasing the ionic strength of the reaction mixture.

In some embodiments, adsorbed proteins may be interrogated by one or more panels of detection antibodies constructed from commercially available panels, for example, available from companies, such as Bio-Techne Corp. (Minneapolis, MN); R&D Systems, Inc. (Minneapolis, MN); and like suppliers.

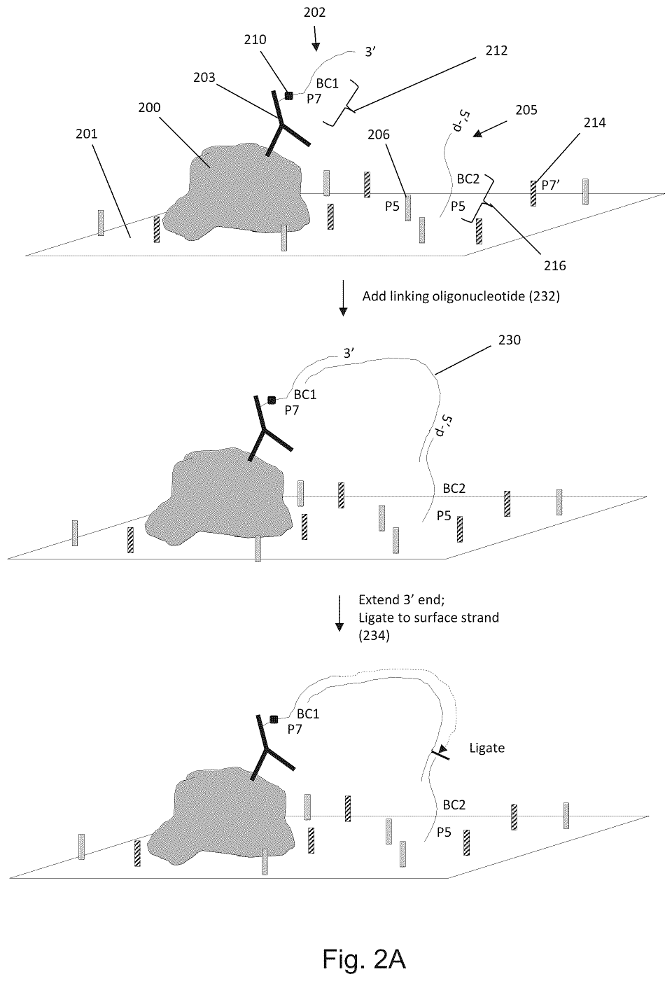

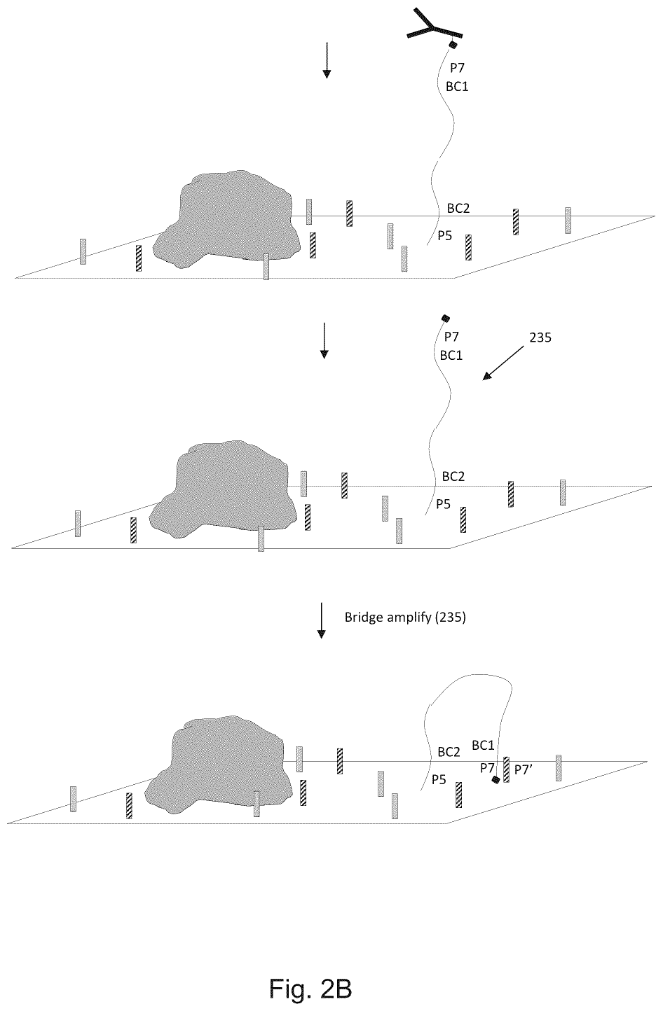

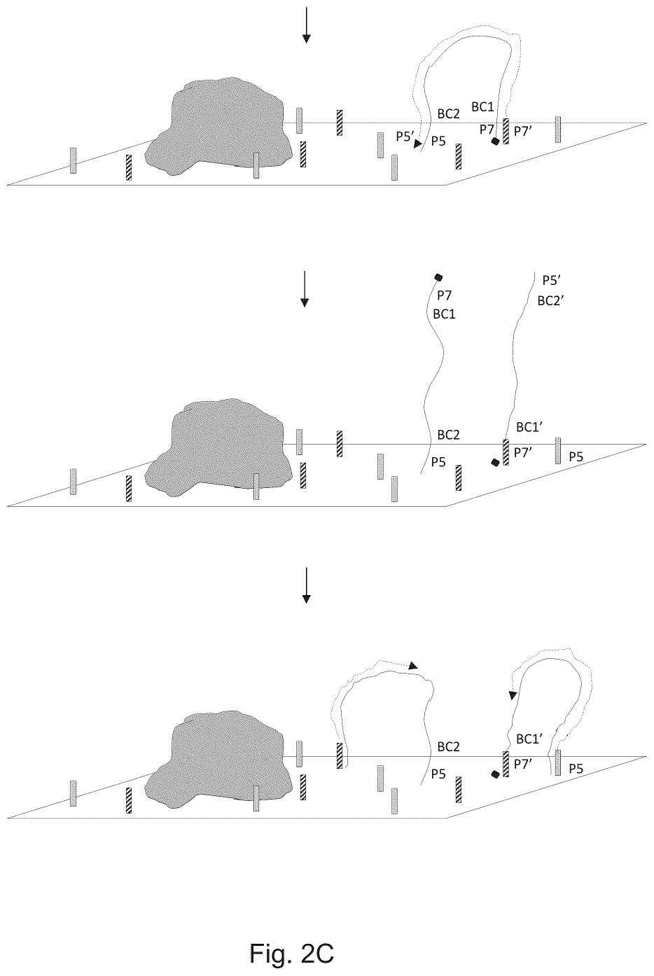

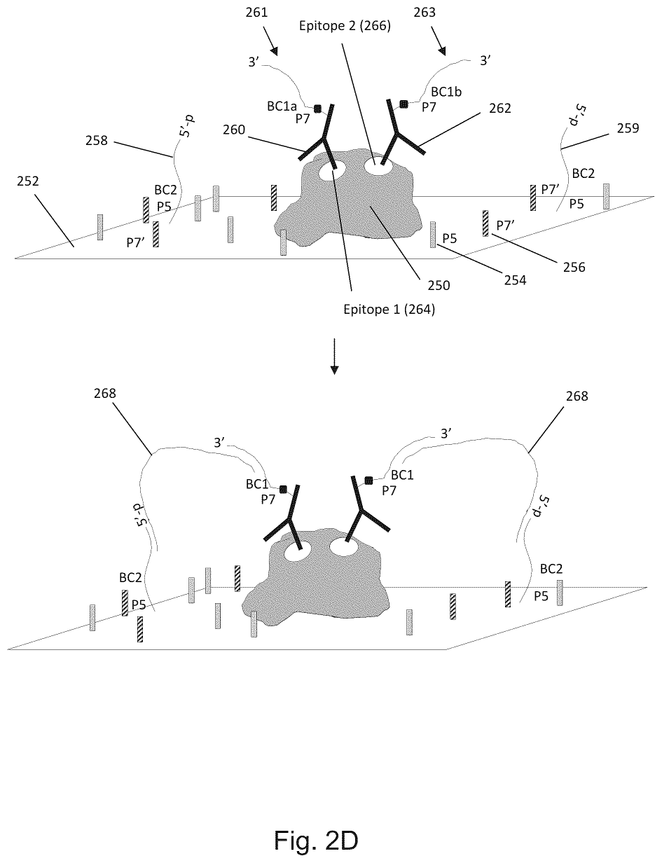

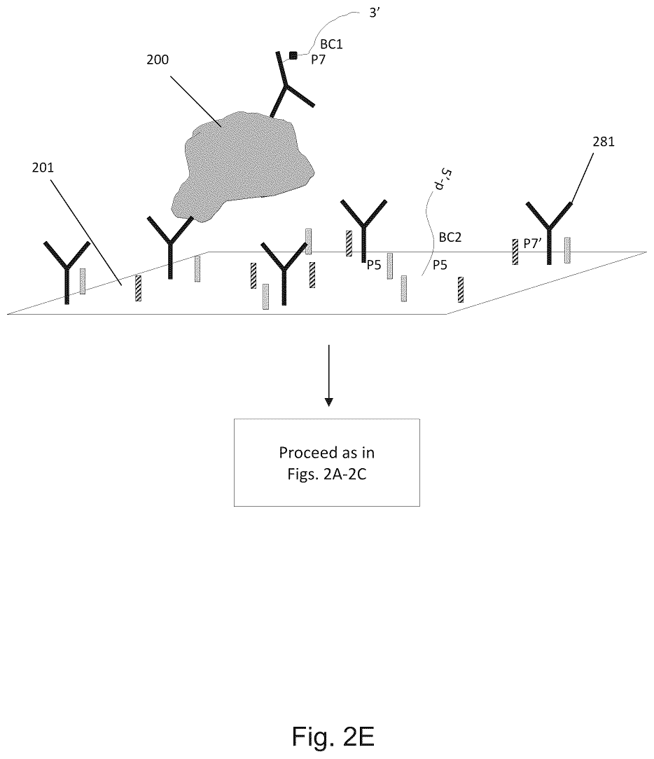

In some embodiments, adsorbed proteins may be detected by the embodiment illustrated in FIGS. 2A-2C. Protein (200) adsorbed on surface (201) is specifically bound by antibody (203) which is linked to the 5′ end of oligonucleotide (205) by cleavable bond (210). Adjacent to its 5′ end, oligonucleotide (205) comprises in order from the 5′ end primer binding site P7 (which may be the same or different than the Illumina, Inc. designated primer binding site) and barcode region (“BC1”) (212). Barcode region BC1 (212) comprises a sequence that uniquely identifies the specific target epitope of antibody (203). Surface (201) further comprises (i) primer oligonucleotides P5 (206) (vertical gray bars, which may be the same or different than the Illumina. Inc., designated primer), (ii) complements to oligonucleotides P7 (i.e. P7′)(214) (vertical striped bars, which may be the same or different than the Illumina, Inc. designated oligonucleotide), and (iii) oligonucleotide strands (205) attached to surface (201) by its 3′ end, having (in order) primer binding site sequence, P5, and barcode “BC2” at its 3′ end (216), and a 5″-phosphate. Barcode region “BC2” (216) comprises a sequence that uniquely identifies a particular region of, or spatial position on, surface (201). After antibody (203) binds to protein (200), the resulting complex is exposed (232) to linker oligonucleotide (230) that hybridizes to both the 3′ end of oligonucleotide (202) and the 5′ end of surface bound oligonucleotide strand (205). After such hybridization, the 3′ end of oligonucleotide (202) is extended (234) by an appropriate polymerase in the presence of deoxynucleoside triphosphates and ligated to the 5′ end of oligonucleotide strand (205). After ligation, linker oligonucleotide (230) may be washed away and cleavable bond (210) cleaved to give strand (235) bound to surface (201), which may be amplified by bridge amplification using the P5 and P7′ primers on surface (201), shown through its first few cycles in FIG. 2C. The resulting amplicons may be sequenced directly (in situ) or cleaved from surface (201) and sequenced by a separate sequencing instrument.

In some embodiments, surface (201) may comprise capture antibodies (281) as illustrated in FIG. 2E, either with P5 and P7′ primers, or without. After capture of protein (200), detection antibody (282) may be added and bound to protein (200) after which it may be used to generate a signal, which may be optical, e.g. fluorescent, or physical, such as, an encoded nucleic acid as described above, or a mixture of the two, e.g. multiplex FISH decoder probes. Multiplex fluorescent in situ hybridization probes (and related decoder probes) are well known to those of skill in the art as evidenced by the following references, which are incorporated herein by reference: Cai et al, U.S. patent publication US2015/0267251; Gunderson et al, U.S. patent publication US2003/0096239; Liehr et al, Histol.Histopathol., 19:229-237 (2004); Gunderson et al, Genomics Research, 14: 870-877 (2004): Kramer, U.S. Pat. No. 7,771,949; Bayani et al, Curr. Protocols in Cell Biology, 24:22.5:22.5.1-22.5.25 (2004); Anderson, chapter 6, Methods in Molecular Biology, vol. 659: 83-96 (2010); and the like.

The some embodiments of the above, proteins (or other target molecules) may be detected on a spatially barcoded surface by the following steps: (a) adsorbing proteins onto a spatially barcoded surface, wherein spatial barcodes on the surface comprise free 5′-phosphates, (b) exposing the spatially barcoded surface under binding conditions to detection antibodies, each detection antibody comprising an antibody barcode that identifies an epitope for which the detection antibody is specific, wherein the antibody barcode comprises a free 3′ end; (c) exposing the spatially barcoded surface under hybridization conditions to a linker oligonucleotide whose 3′ end is configured to hybridize to the free 3′ end of the antibody barcode and the free 5′ end of a spatial barcode on the surface; (d) extending the 3′ end of the antibody barcode to the 5′ end of the spatial barcode; and (e) ligating the 3′ end of the extended antibody barcode to the 5′ end of the spatial barcode. In some embodiments, the 5′ end of the antibody barcode may be cleaved from the detection antibody. In some embodiments, the location and identity of the protein may be determined from the sequences of the extended 3′ end of the antibody barcode and the spatial barcode. In some embodiments, the extended 3′ end of the antibody barcode and the spatial barcode may be amplified. In some embodiments, the extended 3′ end of the antibody barcode and the spatial barcode may be amplified to form clusters that are sequenced using a sequencing-by-synthesis technique. In some embodiments, the extended 3′ end of the antibody barcode and the spatial barcode may be amplified by bridge amplification.

More specific and sensitive measurements of selected adsorbed proteins may be made using the embodiment of FIG. 2D, wherein two antibodies are used in accordance with the embodiment of FIGS. 2A-2C, but the antibodies are specific for separate epitopes on the protein. Since the antibody-identity barcodes will be linked to the same spatial barcode, the embodiment provides the same information as a proximity assay that directly links two antibodies using oligonucleotide hybridization. As above, protein (250) is adsorbed onto surface (252) which comprises P5 primers (254), P7′ primers (256), and oligonucleotide strands (258 and 259) comprising from its 3′ end: a P5 segment, spatial barcode, BC2, and a 5′-phosphate to enabling ligation. After antibodies (260 and 262) bind to epitope 1 (264) and epitope 2 (266), respectively, linker oligonucleotide (268) is added under conditions where the 3′ end of the linker oligonucleotides form duplexes with the 3′ ends of oligonucleotides (261 and 263) of antibodies (260) and 262, respectively) and with the 5′ ends of surface-bound strands (258 and 259). After extension of the 3′ ends of oligonucleotides (261 and 263), ligation of the extended oligonucleotides to strands (258 and 259) and cleaving the cleavable bond to release the antibodies, the resulting oligonucleotide may be amplified and sequenced as described above to identify the two antibodies and the location of the protein they were bound to.

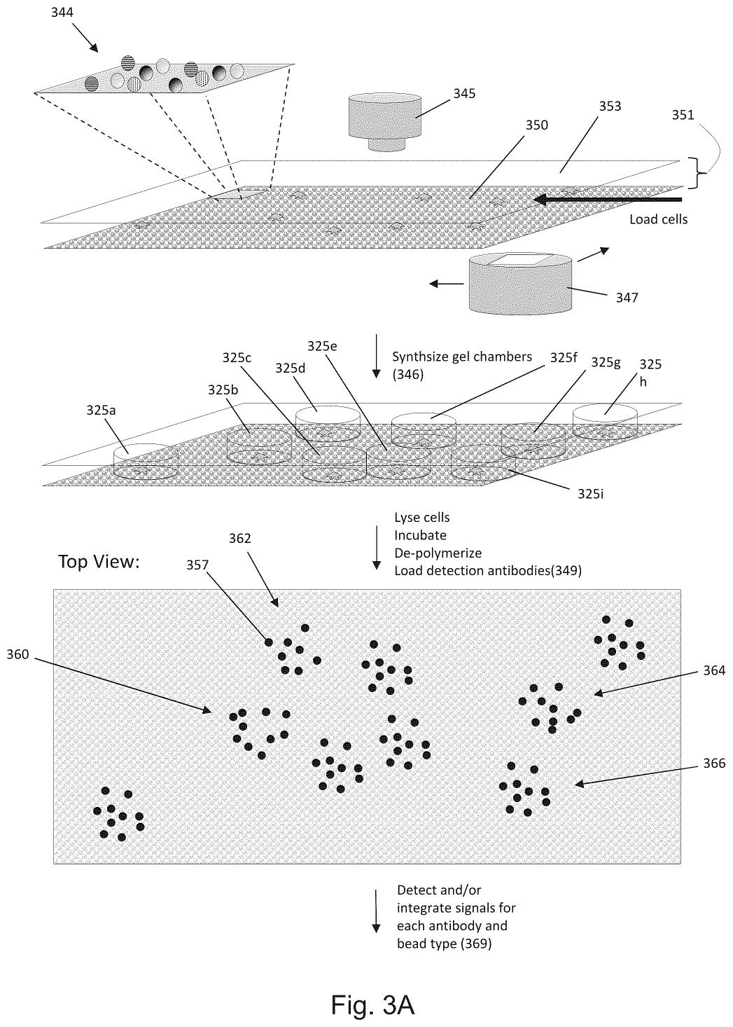

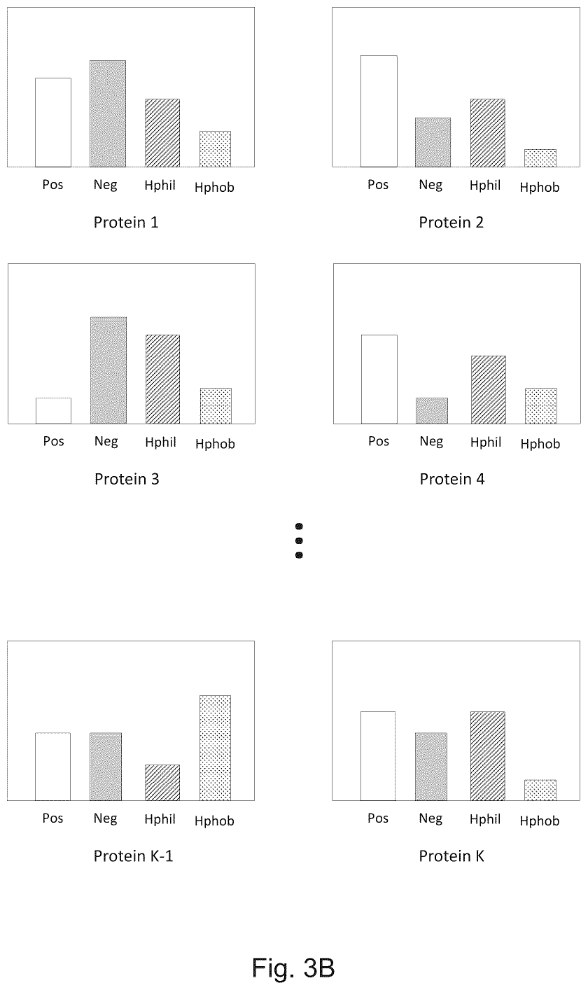

An embodiment in which released proteins may be adsorbed onto a plurality of different surfaces is illustrated in FIGS. 3A-3B. An instrumental arrangement similar to that of FIGS. 1A-1B is provided, except that at least one surface of a channel has disposed thereon a plurality of different beads each comprising a different material that has different surface properties, for example, hydrophobic, hydrophilic, negatively charged, positively charged, or the like. The different bead types may be identified by fluorescent markers (e.g. as with beads available from BioLegends (San Diego)). Surface (350) of channel (351) in the top panel of FIG. 3A represents a surface covered with a closely packed layer of beads, each with different surface properties. This is further illustrated by blow-up (344) of surface (350) where the different bead types are illustrated with different patterns (striped, spotted, solid dark, solid gray). Second surface (353) may be the same as that for the embodiment of FIGS. 1A-1C; namely, a surface of a glass or plastic plate or wall. The functions and operation of detector (345) and light source (347) are as described above. After synthesis (346) of gel chambers, e.g. 325a-i, cells are lysed (349) to release cellular proteins which are adsorbed onto the various bead types enclosed in the chambers along with each cell. After a predetermined incubation time, gel chambers are depolymerized and detection antibodies are loaded into channel (351) where they specifically bind to their target proteins adsorbed onto the surfaces of the different bead types (which for simplicity are shown as clusters (e.g. 360), 362, 364 and 366) of solid spots (e.g. 357), where the locations of the clusters correspond to the locations of the chambers). Each protein will bind to the different surfaces of the beads with a characteristic pattern; namely (for example), a hydrophobic protein will have more representation on a hydrophobic bead surface than on a hydrophilic bead surface, and likewise for other types of bead surfaces. In some embodiments, for each protein, signals may be integrated over each bead type. The results may be displayed as an intensity profile for each protein on each different surface, as illustrated in FIG. 3B.

### Systems and Instrumentation

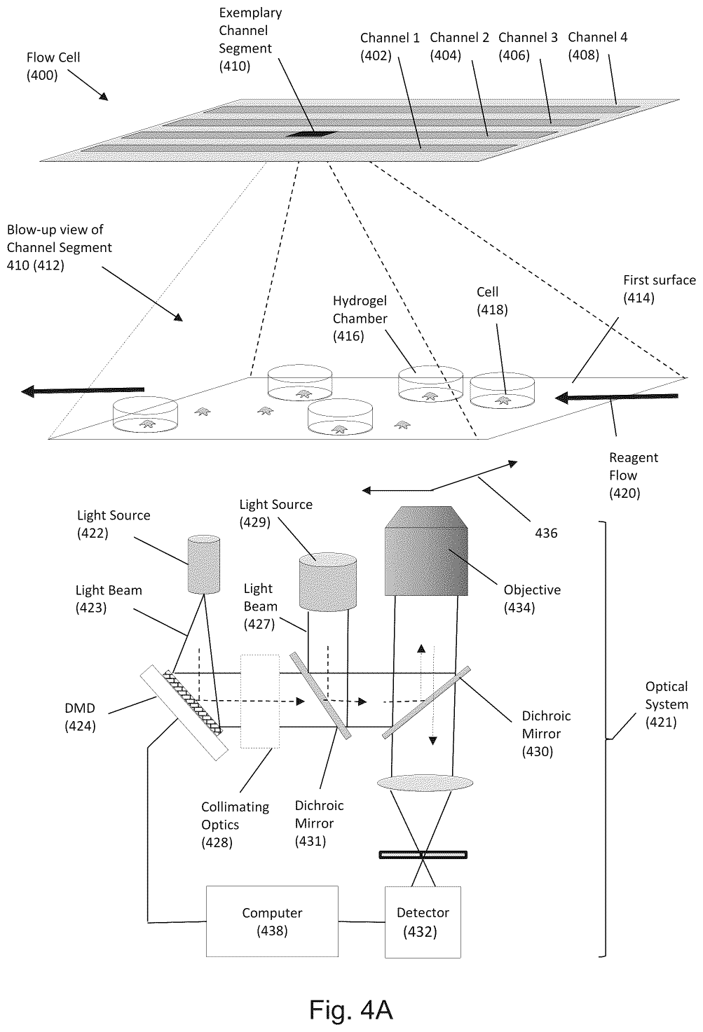

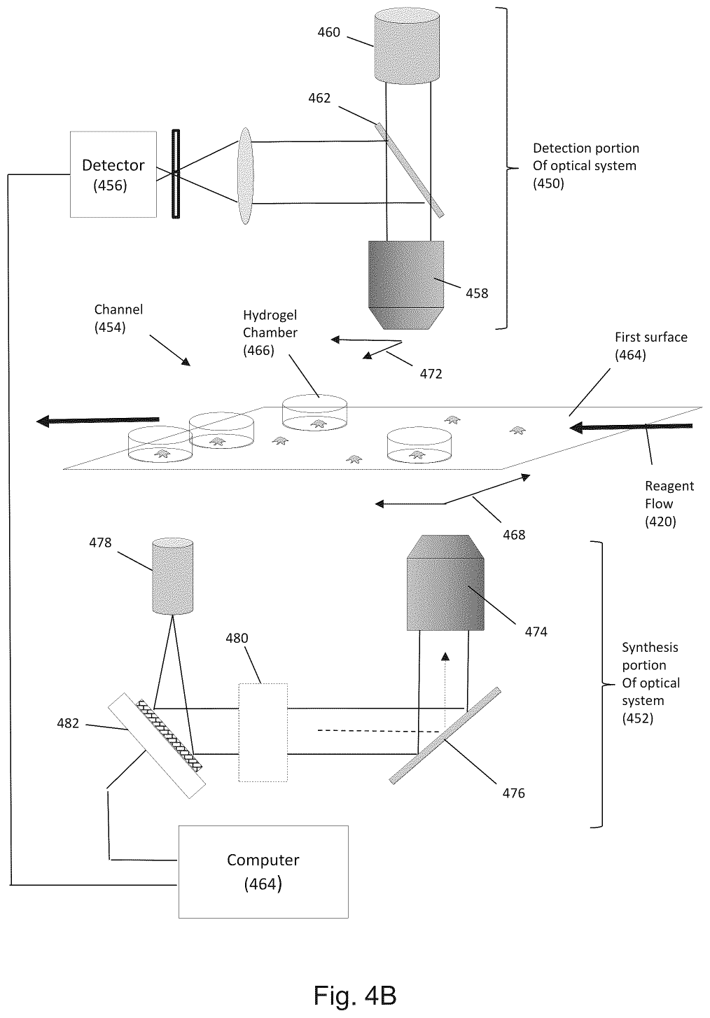

An example of a system for carrying out the above method is illustrated in FIG. 4A. In some cases, flow cell (400) is a component of a fluidic device that provides one or more channels and liquid handling components under programmable control for delivering beads and reagents to the channels. In this illustration, four channels (402, 404, 406, and 408) are shown, with blow-up view (412) of segment (410) of channel 2 (404) shown below. In the abstracted view of flow cell (400) of FIG. 4A. inlets, outlets and other features of the channels are not shown. On first surface (414) of channel 2 (404) a plurality of beads, e.g. (418), can each be enclosed by a hydrogel chamber, e.g. (416). In some embodiments, the porosity of polymer matrix walls of the hydrogel chambers is selected to be impermeable to the beads, but permeable to reagents for forming spatial barcodes. Thus, reagents may be introduced to, and removed from, the interiors of the hydrogel chambers by flowing (420) them through the channels, but beads are retained inside. Below blow-up (412) of channel segment (410) is shown an example of an optical system (421) for photosynthesizing hydrogel chambers at the locations of beads in the channels. One of ordinary skill in the art would recognize that optical systems with different configurations than those of FIG. 4A and 4B may be employed for carrying out these functions. In some embodiments, one or more DMD-objective subsystems for synthesizing hydrogel structures may be employed to increase the speed of synthesis by synthesizing multiple structures simultaneously.

Returning to FIG. 4A, for photosynthesizing the hydrogel chambers, light source (422) can generate a light beam (423) of appropriate wavelength light (e.g. UV light) that passes through an appropriate photo-mask or beam-shaping or beam steering (Galvo) system for shaping a beam to synthesize a desired structure or structures in a channel. In some embodiments, a digital micromirror device (DMD)(424) is employed, in other embodiments, a physical photo-mask may be employed. Chamber position, shape and polymer matrix wall thickness can be determined at least in part from bead position information determined from images collected by detector (432). Reflected light from DMD (424) can be shaped using conventional optics, e.g. collimating optics (428), and can be directed through objective lens system (434) into channel 2 segment (410). Objective (434) and flow cell (400) may move relative to one another in the xy-directions (436) to photosynthesize chambers at any position in any of the channels. In some embodiments, flow cell (400) moves and optical system (421) is stationary. In some embodiments, objective (434) may also direct light beam (427) from light source (429) to targets, such as cells, on first surface (414) and collect optical signals, such as fluorescent signals, from assays taking place on first surface (414). Alternatively, optical signal collection may be carried out with a separate objective as shown if FIG. 4B. Information collected by detector (432), or its counterpart in the embodiment of FIG. 4B, particularly cellular positions in their respective channels, is employed by computer (438) and/or subsidiary controllers to direct DMD (424) and translation devices controlling the relative positions of objective (434) and flow cell (400) to synthesize hydrogel chambers of the appropriate shape and size at the appropriate locations.

FIG. 4B illustrates an alternative optical system in which the detection portion (450) of the optical system moves (472) independently from the movement (468) of the synthesis portion (452) of the optical system. Detection portion (450) of the optical system may comprise detector (456), objective (458), light source (460) and interconnecting optical elements, such as dichroic mirror (462). As with the embodiment of FIG. 4A, detector (456) can be operationally associated with computer (464) and the synthesis portion (452) of the optic system to provide synthesis portion (452) with bead position information. Computer (464) and (438) can also be operationally associated with stages and/or motors controlling the relative positions of the objectives of the optical systems and the position of the flow cell. In this embodiment, synthesis portion (452) of the optical system is located on the other side of first surface (464) from detection portion (450). As with the embodiment of FIG. 4A, the synthesis portion (452) comprises the conventional components objective (474), mirror (476), collimating optics (480), DMD (482) and light source (478).

In some embodiments, systems for implementing the methods described herein comprise (i) a channel comprising a surface, (ii) a spatial energy modulation element in optical communication with the surface, and (iii) a detector in optical communication with the surface and in operable association with the spatial energy modulating element, the detector identifying cells and determining positions thereof on the surface. It is understood that the term “detector” as used herein may include, but not be limited by, a microscope element that collects and optionally magnifies an image of a portion of a channel and an image analysis element that comprises software for identifying cells and associated position information. A computer element can use such information generated by a detector together with user input to generate commands for other elements, such as, the spatial energy modulating element to carry out a variety of functions including, but not limited to, synthesizing chambers, “on-demand” degrading of chambers, selectively photo-degrading chambers, and the like. Examples of configurations of such embodiments are illustrated in FIGS. 4A-4B which are described above. In some embodiments, a channel of a fluidic device further comprises a second surface (e.g. illustrated in FIG. 2) wherein said first surface and the second surface are disposed opposite one another across the channel, and wherein the polymer matrix walls of the chambers extend from the first surface to the second surface to form chambers each having an interior. In some embodiments, chambers in a channel each enclose a single cell. In some embodiments, both the first wall and the second wall are made of optically transmissive materials, such as, glass, plastic, or the like, and are positioned so that the first surface and second surface are substantially parallel to one another. The perpendicular distance between a first surface and a second surface may be in the range of from 10 μm to 500 μm, or in the range of from 50 μm to 250 μm.

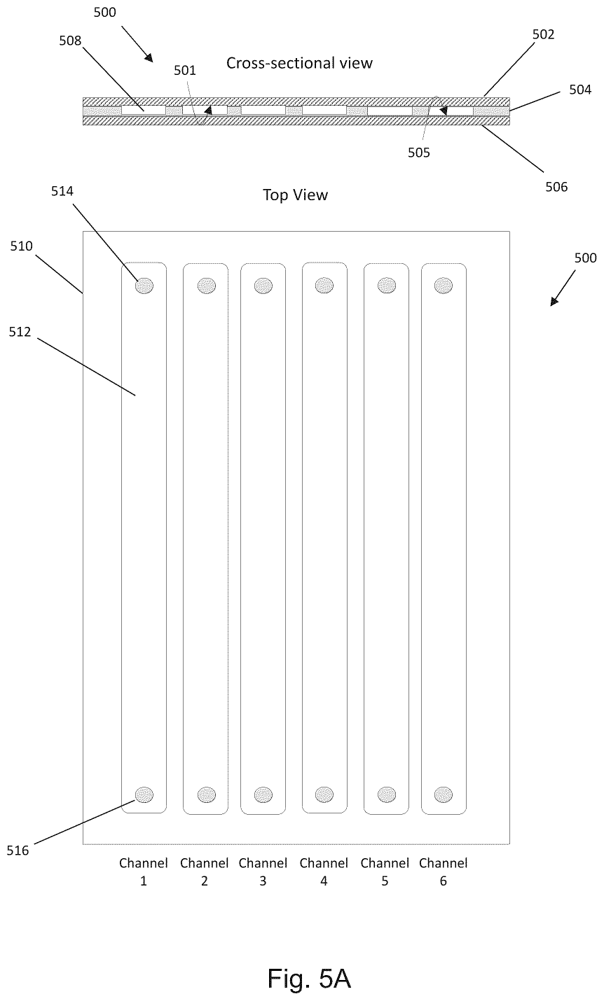

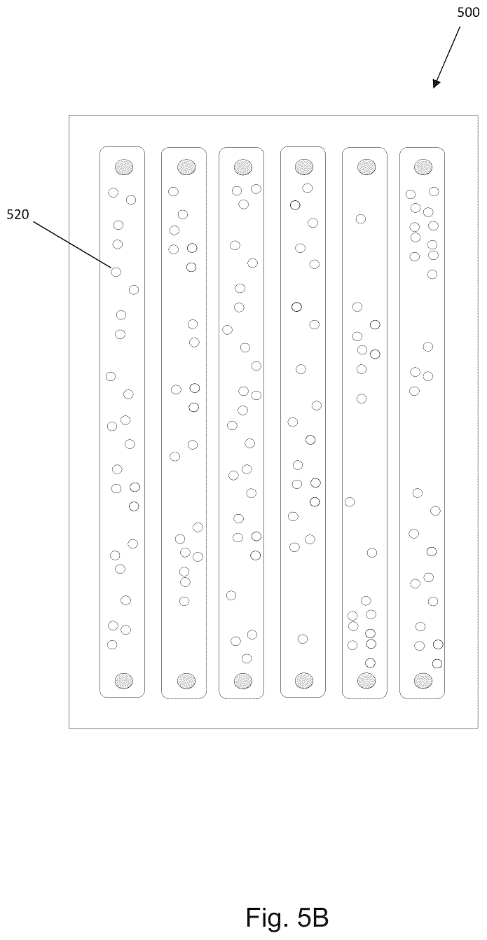

In some embodiments, a plurality of channels may be arranged together in a flow channel as illustrated in FIGS. 5A-5B. In some embodiments, the plurality of channels may be in the range of from 2 to 12, or from 2 to 8, or from 2 to 6, or in the range of from 2 to 4. An example of a flow cell (500) is shown in a cross-sectional view and a top view. In some cases, flow cell (500) has bottom, or first, wall (506) with first surface (505); top, or second, wall (502) with second surface (501); and sandwiched sealingly therebetween spacer (504) whose longitudinal holes form channels 1-6, one of which is indicated by (508) in the cross-sectional view; and by (512) in the top view. In some embodiments, spacer (504) may have a thickness in the range of from 10 m to 500 m, or in the range of from 50 m to 250 m, which determines the interior height of the channels. Top wall (502) comprises inlets (514) and outlets (516) for either separately or jointly loading and removing reagents and beads from channels 1-6. In some embodiments, at least one of walls (502) and (506) are made of light transmissive materials, such as glass, plastic, or the like. Flow cell (500) may be operationally associated with a fluidic device that delivers reagents and beads to any of channels 1-6 under programmed control. Guidance for particular designs, including fluid handling and valving for such fluidic systems may be found in U.S. Pat. Nos. 8,921,073; 8,173,080; 8,900,828; and the like, which are incorporated herein by reference. FIG. 5B illustrates channels of flow cell (500) with random distributions (not to scale) of hydrogel chambers with annulus-like cross-sections, such as (520), on their first surfaces.

As noted above, any of first surfaces, second surfaces or polymer matrix wall of chambers may comprise capture elements and other functional groups for carrying out a variety of operations including, but not limited to, capturing beads, capturing cells, capturing analytes (such as, mRNA, secreted proteins, intracellular proteins, or genomic sequences), capturing constituents of analytical reagents (such as, oligonucleotide labels from antibodies), and the like. Derivatizing surfaces for such purposes is well-known to those skilled in the art, as evidenced by the following references: Integrated DNA Technologies brochure (cited above); Hermanson (cited above); and the like.

As noted above, in some embodiments, a fluidic device of the method comprises or is operationally associated with a detector that either may share an optical path of the spatial energy modulating element or may be disposed adjacent to the second wall or opposite the first wall from the spatial energy modulating element in embodiments, such as wells, that have only a first wall and first surface. The detector is positioned so that it is capable of detecting optical signals from or adjacent to cells in the channel, for example, distributed over the first surface in chambers. In some embodiments, the first and second walls each comprise optically transmissive material, for example, so that a spatial energy modulating element may project light energy to the interior of the channel, and so that a detector may detect optical signals, such as fluorescent emissions or reflected light from biological components. In some embodiments, the projected energy from the spatial energy modulating element is a light energy from a light beam. In some embodiments, the light beam projected by the spatial energy modulating element may have a complex cross-section that permits (in various embodiments) the simultaneous synthesis of a plurality of chambers. Optically transmissive materials include, but are not limited to, glass, quartz, plastic, and like materials.

Spatial energy modulating elements using light energy for polymerization may comprise physical photomasks or virtual photomask, such as, a digital micromirror device (DMD). The following references, which are hereby incorporated by reference, provide guidance in selecting and operating a DMD for photopolymering gels: Chung et al, U.S. Pat. No. 10,464,307; Hribar et al, U.S. Pat. No. 10,351,819; Das et al, U.S. Pat. No. 9,561,622; Huang et al, Biomicrofluidics, 5: 034109 (2011); and the like.

As used herein, “channel” generally means a container capable of holding fluid (which may be static or flowing) and having at least one surface on which beads may be disposed and chambers synthesized. In some embodiments, a channel may have a first surface and/or a second surface on which chambers may be synthesized and/or on which beads or particles may be disposed. As used herein, reference to a “surface” without reference to “first” or “second” is intended to comprise a first surface or a second surface (if two are present in a fluidics device, e.g. comprising a flow cell). In some embodiments, a channel may constrain a flow of fluid therethrough from an inlet to an outlet. In other embodiments, a channel may comprise a non-flowing volume of fluid that may be removed, replaced or added to by way of an opening or inlet; that is, in some embodiments, a channel may be a well or a well-like structure.

### Gel Chambers

A wide variety of photo-synthesizable gels and degradable gels are available for implementing the methods described herein. Guidance for selecting such gels for desired properties including, but not limited to, biocompatibility, porosity, gelation speed, degradation speed, and like properties, is provided in the following references, which are incorporated by reference: Kharkar et al, Chem. Soc. Rev., 42: 7335-7372 (2013); Kharkar et al Polymer Chem., 6(31): 5565-5574 (2015); Neumann et al, Acta Biomater., 39: 1-11 (2016); DeForest et al, Nature Chemistry, 3(12): 925-931 (2012); Bowman et al, U.S. Pat. No. 9,631,092; LeValley et al, ACS Appl. Bio. Mater., 3(10): 6944-6958 (2020); Kabb et al, ACS Appl. Mater. Interfaces, 10: 16793-16801 (2018); Fairbanks et al, Macromolecules, 44: 2444-2450) (2011); Fairbanks et al, Adv. Mater., 21(48): 5005-5010 (2009); Sugiura et al, U.S. patent publication US2016/0177030; Shih et al, Biomacromolecules, 13(7): 2003-2012 (2012); and the like. In some embodiments, photo-synthesized gels are formed using a photo-initiator for radical polymerization. In some embodiments, photo-initiators comprise Irgacure 2959, Lithium phenyl-2,4,6-trimethylbenzoylphosphinate (LAP), or Eosin-Y (e.g. see Choi et al, Biotechniques, 66(1): 40-53 (2019)). In some embodiments, hydrogel precursors comprise hyaluronic acid, chitosan, heparin, alginate, polyethylene glycol (PEG), multi-arm PEG, poly(ethylene glycol)-b-poly(propylene oxide)-b-poly(ethylene glycol) (PEG-PPO-PEG), poly(lactic acid-co-glycolic acid)-b-poly(ethylene glycol)-b-poly(lactic acid-co-glycolic acid) (PLGA-PEG-PLGA), and poly(vinyl alcohol). In some embodiments, polymer precursors comprise PEG or multi-arm PEG. In some embodiments, polymer precursors comprise an enzymatically degradable cross-linker. In some embodiments, such enzymatically degradable cross-linker is degradable by an esterase or a peptidase. In some embodiments, polymer precursors comprise a photo-degradable cross-linker. In some embodiments, such photo-degradable cross-linker comprises a nitrobenzyl group.

In some embodiments, such photo-degradable cross-linker comprises a courmarin moiety. In some embodiments, photo-degradable hydrogels are used, for example, because photo-degradation of hydrogel chambers may be carried out selectively and on-demand, so that specified hydrogel chambers may be degraded without affecting non-selected hydrogel chambers are unaffected. In some embodiments, hydrogel chambers are degraded non-selectively, so that all hydrogel chambers in a given channel (or other vessel) are degraded simultaneously. In some embodiments, such non-selective degradation is carried out with a cleavage reagent that specifically cleaves a labile bond in a hydrogel. For example, such cleavage agent comprises a reducing agent. In some embodiments, such non-specific degradation is carried out with an enzyme that cleaves a bond or chemical element in a hydrogel. Chemical elements may include, but are not limited to, peptides, polysaccharides and oligonucleotides.

In the figures, for convenience, hydrogel chambers are illustrated as standing in isolation without connection with adjacent chambers and as having a cylindrical or annular-like shapes; however, a spatial energy modulating element may synthesize chambers of different shapes and sizes, as is useful for particular applications. In some embodiments of the proliferation assay, each hydrogel chamber synthesized has the same shape and area, for example, annular-like with an interior area selected from the range of 0.001 to 0.01 mm².

Function. A wide variety of photo-synthesizable gels may be used in connection with the methods described herein. In some embodiments, hydrogels are used in particular because of their compatibility with living cells and the versatility of formulating gels with desired properties including, but not limited to, porosity, degradability, mechanical strength, ease and speed of synthesis, and the like. In some embodiments, gels or hydrogels are both photo-synthesizable and photo-degradable. In some embodiments, gel degradation mechanisms are compatible with living cells.

Porosity. In some embodiments, hydrogel porosity is selected to permit passage of selected reagents while at the same time preventing the passage of other reagents or objects, such as, a cell or proteins of a lysed cell. In some embodiments, crosslinking the polymer chains of the hydrogel structure forms a hydrogel matrix having pores (i.e., a porous hydrogel matrix). In some embodiments, the pores have an average diameter of from about 2 nm to about 25 nm, or from about 5 nm to about 20. In some embodiments, average pore diameters are selected to prevent the passage of cellular proteins. In some embodiments, average pore diameters are selected to prevent the passage of cellular proteins having a molecular weight of 1 kiloDaltons or greater. In some embodiments, average pore diameters are selected to prevent the passage of cellular proteins having a molecular weight of 5 kiloDaltons or greater. In some embodiments, the pore size of the hydrogel structures is tuned by varying the ratio of the concentrations of polymer precursors to the concentration of crosslinkers, varying pH, salt concentrations, temperature, light intensity, and the like. Guidance for selecting materials and conditions to control hydrogel porosity may be found in the following references: Jung et al, Biochem. Eng. J., 135: 123-132 (2018); Winther et al, Biochim. Biophys. Acta, 1840(2): doi:10.1016/j.bbagen.3013.03.031 (2014); Annabi et al, Tissue Engineering, part B, 16(4):371-383 (2010); and the like.

Size and Shape of Hydrogel Chambers. In some embodiments, a polymer matrix wall of a chamber inhibits passage of a predetermined component, such as a mammalian cell, a bacterial cell, or proteins from a lysed cell. In some embodiments, a polymer matrix wall extends from the first surface to a second surface (parallel to the first surface) to form a chamber within a channel. In some embodiments, a chamber has polymer matrix walls and an interior. In some embodiments, the interior of a chamber is sized for enclosing a cell, such as a mammalian cell. For example, such chamber may comprise a cylindrical shell or a polygon shell, comprising an inner space, or interior and a polymer matrix wall. In some embodiments, such chambers may have annular-like cross-sections. As used herein, the term “annular-like cross-section” means a cross-section topologically equivalent to an annulus. In some embodiments, the inner space, or interior, of a chamber has an inner diameter from 1 μm to 500 μm and a volume in the range of from 1 pico liter to 200 nano liters, or from 100 pico liters to 100 nano liters, or from 100 picoliters to 10 nano liters. In some embodiments, the polymer matrix wall has a thickness of at least 1 μm (micrometer). In some embodiments, the height of a chamber with an annular-like cross section have a value in the range of from 10 m to 500 m, or in the range of from 50 m to 250 m. In some embodiments, a polymer matrix wall having an annular-like cross-section has an aspect ratio (i.e., height/width) of 1 or less. In some embodiments, aspect ratio and polymer matrix wall thickness are selected to maximize chamber stability against forces, such as reagent flow through the channel, washings, and the like. In some embodiments, the at least one polymer matrix wall is a hydrogel wall. In some embodiments, the at least one polymer matrix is degradable. In some embodiments, the degradation of the at least one polymer matrix is “on demand.”

In some embodiments, chambers in a channel are non-contiguous. In some embodiments, chambers in a channel may be contiguous with adjacent chambers. In some embodiments, chambers may share polymer matrix walls with one another. In some embodiments, chambers may be synthesized with slits or other orifaces large enough to permit passage of certain components, e.g. beads, but small enough to prevent passage of other components, e.g. cells.

Hydrogel Compositions. As mentioned above, hydrogel compositions may vary widely and hydrogels may be formed by a variety of methods. Biocompatible hydrogel precursors comprise, but are not limited to, hyaluronic acid, chitosan, heparin, alginate, polyethylene glycol (PEG), multi-arm PEG, poly(ethylene glycol)-b-poly(propylene oxide)-b-poly(ethylene glycol) (PEG-PPO-PEG), poly(lactic acid-co-glycolic acid)-b-poly(ethylene glycol)-b-poly(lactic acid-co-glycolic acid) (PLGA-PEG-PLGA), and poly(vinyl alcohol). In some embodiments, hydrogels are formed by photo-initiated free radical crosslinking. In some embodiments, hydrogels are formed by photo-initiated thiol-ene reactions.

Hydrogel Degradation. In some embodiments, hydrogel chambers are degradable or depolymerizable either generally within a channel or “on demand” within a channel. Hydrogel chambers that are generally degradable are degraded by treatment with a degradation agent, or equivalently, a depolymerization agent that is exposed to all chambers within channel. Depolymerization agents may include, but are not limited to, heat, light, and/or chemical depolymerization reagents (also sometimes referred to a cleaving reagents or degradation reagents). In some embodiments, on demand degradation may be implemented using polymer precursors that permit photo-crosslinking and photo-degradation, for example, using different wavelengths for crosslinking and for degradation. For example, Eosin Y may be used for radical polymerization at defined regions using 500 nm wavelength, after which illumination at 380 nm can be used to cleave the cross linker. In other embodiments, photo-caged hydrogel cleaving reagents may be included in the formation of polymer matrix walls. For example, acid labile crosslinkers (such as esters, or the like) can be used to create the hydrogel and then UV light can be used to generate local acidic conditions which, in turn, degrades the hydrogel. In some embodiments, the at least one polymer matrix is degradable by at least one of: (i) contacting the at least one polymer matrix with a cleaving reagent; (ii) heating the at least one polymer matrix to at least 90° C.; or (iii) exposing the at least one polymer matrix to a wavelength of light that cleaves a photo-cleavable cross linker that cross links the polymer of the at least one polymer matrix. In some embodiments, the at least one polymer matrix comprises a hydrogel. In some embodiments, the cleaving reagent degrades the hydrogel. In some embodiments, the cleaving reagent comprises a reducing agent, an oxidative agent, an enzyme, a pH based cleaving reagent, or a combination thereof. In some embodiments, the cleaving reagent comprises dithiothreitol (DTT), tris(2-carboxyethyl)phosphine (TCEP), tris(3-hydroxypropyl) phosphine (THP), or a combination thereof. In some embodiments, the surface of the polymer matrix or hydrogel may be functionalized by coupling a functional group to the polymer matrix or hydrogel. Some nonlimiting examples of functional group may include a capture reagent (e.g., pyridinecarboxaldehyde (PCA)), an acrylamide, an agarose, a biotin, a streptavidin, a strep-tag II, a linker, a functional group comprising an aldehyde, a phosphate, a silicate, an ester, an acid, an amide, an aldehyde dithiolane, PEG, a thiol, an alkene, an alkyne, an azide, or a combination thereof. In some cases, the functionalized polymer matrix may be used to capture biomolecules inside a polymer matrix compartment formed adjacent to (e.g., around or on) the biological component.

The biomolecule may be produced by the biological component (e.g., secretome from a cell). The functionalized surface of the polymer matrix inside the compartment may be used to capture reagents or molecules from outside the compartment. The functionalized surface may increase surface area covered by a reagent, a molecular sensor, or any molecule of interest (e.g., an antibody).

Photosynthesis. In some embodiments, the generation of a polymer matrix within a channel or well of a fluidic device comprises exposing the one or more polymer precursors to an energy source. In some embodiments, the energy source is a light generating device. In some embodiments, the light generating device generates light at 350 nm to 800 nm. In some embodiments, the light generating device generates light at 350 nm to 600 nm. In some embodiments, the light generating device generates light at 350 nm to 450 nm. In some embodiments, the light generating device generates UV light. In some embodiments, the generation of a polymer matrix within said fluidic device is performed using a spatial light modulator (SLM) (i.e. a spatial energy modulation element that is capable of generating desired light intensity pattern spatially). In some embodiments, the SLM is a digital micromirror device (DMD). In some embodiments, the SLM is a laser beam steered using a galvanometer. In some embodiments, the SLM is liquid-crystal based.

While the present disclosure has been described with reference to several particular example embodiments, those skilled in the art will recognize that many changes may be made thereto without departing from the spirit and scope of the systems and methods described herein. The present disclosure is applicable to a variety of sensor implementations and other subject matter, in addition to those discussed above.

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immuology, 6^{th} edition (Saunders, 2007).

“Antibody” as used herein generally means any binding compound capable of specifically binding to a given protein including, without limitation, immunoglobulin molecules or fragments thereof and aptamers. Fragments of immunoglobulin molecules include, but are not limited to, Fab, Fv and F(ab′)2, Fab′ fragments, and the like.

“Detection antibody” as used herein generally means an antibody conjugated with a detection moiety that permits the identification of a protein that the antibody is bound to. A detection moiety may comprise a fluorescent dye, a barcode, an enzyme, or the like. In some embodiments, a detection moiety may be covalently linked to an antibody. In some embodiments, a detection moiety may be conjugated to an antibody by a scissile bond or by hydridization.

“Cells” as used herein generally refer to biological cells that may be assayed by methods and systems described herein, but are not limited to, vertebrate, non-vertebrate, eukaryotic, mammalian, microbial, protozoan, prokaryotic, bacterial, insect, or fungal cells. In some embodiments, mammalian cells are assayed by methods and systems described herein. In particular, any mammalian cell which may be, or has been, genetically altered for use in a medical, industrial, environmental, or remedial process, may be analyzed by methods and systems described herein. In some embodiments, “cells” as used herein comprise genetically modified mammalian cells. In some embodiments, “cells” comprise stem cells. In some embodiments, “cells” refer to cells modified by CRISPR Cas9 techniques. In some embodiments, “cells” refer to cells of the immune system including, but not limited to, cytotoxic T lymphocytes, regulatory T cells, CD4+ T cells, CD8+ T cells, natural killer cells, antigen-presenting cells, or dendritic cells. Of special interest are cytotoxic T lymphocytes engineered for therapeutic applications, such as cancer therapy.

“Hydrogel” as used herein generally means a gel comprising a crosslinked hydrophilic polymer network with the ability to absorb and retain large amounts of water (for example, 60 to 90 percent water, or 70 to 80 percent) without dissolution due to the establishment of physical or chemical bonds between the polymeric chains, which may be covalent, ionic or hydrogen bonds. Hydrogels exhibit high permeability to the oxygen and nutrients, making them attractive materials for cell encapsulation and culturing applications.

Hydrogels may comprise natural or synthetic polymers and may be reversible (i.e. degradable or depolymerizable) or irreversible. Synthetic hydrogel polymers may include polyethylene glycol (PEG), poly(2-hydroxyethyl methacrylate) and poly(vinyl alcohol). Natural hydrogel polymers include alginate, hyaluronic acid and collagen. The following reference describe hydrogels and their biomedical uses: Drury et al, Biomaterials, 24: 4337-4351 (2003); Garagorri et al, Acta Biomatter, 4(5): 1139-1147 (2008); Caliari et al, Nature Methods, 13(5): 405-414 (2016); Bowman et al, U.S. Pat. No. 9,631,092; Koh et al, Langmuir, 18(7): 2459-2462 (2002).

“On demand” as used herein generally means an operation may be directed to individual, discrete, selected locations (e.g. a spatial location of polymer precursor solution; or a selected polymer matrix chamber). Such selection may be based on manual observation of optical signals or data collected by a detector, or such selection may be based on a computer algorithm operating on optical signals or data collected by a detector. Manual observation of optical signals or data collected by a detector can include either real-time detection or detection at a time period prior to modulating a unit of energy to polymerize polymer precursors or degrading a chamber. For example, a subset of chambers (all formed with photo-degradable polymer matrix walls) may be pre-selected for releasing and removing their contents based on position information and the values of optical signals from an analytical assay carried out in the chambers. The pre-selected chambers may be photo-degraded by selectively projecting a light beam of appropriate wavelength characteristics (for example, with the spatial energy modulating element) to degrade the polymer matrix walls of the pre-selected chambers. In another example, a plurality of chambers may be observed in real-time (e.g. via fluorescent microscopy) for detection of an analyte of interest and one or more chambers of the plurality of chambers is selected, in real-time, upon detection of the analyte of interest, for degradation.

“Polymer matrix” as used herein generally refers to a phase material (e.g. continuous phase material) that comprises at least one polymer. In some embodiments, the polymer matrix refers to the at least one polymer as well as the interstitial space not occupied by the polymer. A polymer matrix may be composed of one or more types of polymers. A polymer matrix may include linear, branched, and crosslinked polymer units. A polymer matrix may also contain non-polymeric species intercalated within its interstitial spaces not occupied by polymer chains. The intercalated species may be solid, liquid, or gaseous species. For example, the term “polymer matrix” may encompass desiccated hydrogels, hydrated hydrogels, and hydrogels containing glass fibers. A polymer matrix may comprise a polymer precursor, which generally refers to one or more molecules that upon activation can trigger or initiate a polymeric reaction. A polymer precursor can be activated by electrochemical energy, photochemical energy, a photon, magnetic energy, or any other suitable energy. As used herein, the term “polymer precursor” includes monomers (that are polymerized to produce a polymer matrix) and crosslinking compounds, which may include photo-initiators, other compounds necessary or useful for generating polymer matrices, especially polymer matrices that are hydrogels.

What is claimed is:

1. A method of determining proteomes of single cells, comprising:
(a) synthesizing one or more gel chambers enclosing each of one or more cells disposed on or adjacent to a surface of a channel;
(b) lysing the one or more cells so that cellular proteins of the one or more cells are released into the one or more gel chambers and at least a portion of the cellular proteins are adsorbed onto the surface enclosed by the one or more gel chambers; and
(c) detecting the adsorbed cellular proteins with detection antibodies.

2. The method of claim 1, wherein a fluidic device comprises the surface of the channel, wherein the surface is a top surface or bottom surface of the channel.

3. The method of claim 1, wherein the cellular proteins or the portion of the cellular proteins adsorb to a material coupled to the surface.

4. The method of claim 1, wherein the cellular proteins or the portion of the cellular proteins bind to one or more capture antibodies coupled to the surface.

5. The method of claim 1, further comprising collecting optical signals from the one or more cells disposed on or adjacent to the surface; and determining, prior to the synthesizing, the position of each of the one or more cells from the optical signals.

6. The method of claim 1, further comprising depolymerizing the one or more gel chambers prior to the detecting.

7. The method of claim 6, further comprising de-adsorbing the adsorbed cellular proteins prior to the depolymerizing.

8. The method of claim 7, wherein the de-adsorbed cellular proteins diffuse into the interior of their gel chamber and re-adsorb to the surface prior to the depolymerizing.

9. The method of claim 1, wherein the surface is a surface of a particle in the channel.

10. The method of claim 1, wherein the surface comprises particles of a plurality of types disposed thereon, wherein each type of particle comprises a surface with different protein adsorption characteristics.

11. The method of claim 10, wherein the particles of each type have a size and a quantity such that each of the one or more gel chambers encloses particles of every type.

12. The method of claim 10, wherein the one or more gel chambers are impermeable to the particles.

13. The method of claim 1, wherein each of the detection antibodies comprises a label capable of generating a signal indicative of a protein which such detection antibody is specific for.

14. The method of claim 13, wherein the label is coupled to the detection antibody by a scissile bond or by hybridization to an oligonucleotide coupled to the detection antibody.

15. The method of claim 1, wherein the one or more gel chambers are permeable to the detection antibodies.

16. The method of claim 15, wherein the method further comprises inputting the detection antibodies into the one or more gel chambers to the cellular proteins adsorbed onto the surface enclosed by the one or more gel chambers.

17. The method of claim 1, further comprising: prior to (a), using a detector in optical communication with the surface to collect one or more optical signals for identifying the one or more cells and positions thereof on the surface; wherein positions of the one or more gel chambers in the channel are determined by the positions of the one or more cells identified by the detector.

18. The method of claim 17, wherein the synthesizing comprises projecting light into the channel with a spatial energy modulating element in operable communication with the detector, such that the projected light causes cross-linking of one or more polymer precursors to form the one or more gel chambers.

19. The method of claim 13, wherein the signal is an optical signal.

20. The method of claim 13, wherein the signal comprises a nucleic acid barcode.

21. The method of claim 17, further comprising using the detector to collect one or more optical signals for identifying positions of particles in the channel, wherein positions of the one or more gel chambers in the channel are at least in part determined by the positions of the particles identified by the detector.

22. The method of claim 1, wherein each detection antibody comprises an antibody barcode that identifies an epitope for which the detection antibody is specific, wherein the surface of the channel or an additional surface of the channel comprises spatial barcodes, and wherein the detecting comprises:
(i) hybridizing linker oligonucleotides to the antibody barcodes and spatial barcodes, thereby coupling the antibody barcodes to the spatial barcodes,
(ii) ligating the antibody barcodes to the spatial barcodes, and
(iii) sequencing the antibody barcodes and the spatial barcodes.

23. The method of claim 22, further comprising extending the antibody barcodes over the linker oligonucleotides prior to (ii).

24. The method of claim 22, wherein the spatial barcodes comprise sequences associated with locations of the spatial barcodes on the surface of the channel or the additional surface of the channel.

25. The method of claim 22, wherein the surface of the channel comprises the spatial barcodes.

26. The method of claim 22, wherein the locations and identities of the cellular proteins are determined from nucleotide sequences of the antibody barcode and the spatial barcode.

27. The method of claim 1, wherein the one or more chambers comprise annular-like cross-sections.

28. The method of claim 1, wherein the one or more chambers each enclose a single cell of the one or more cells.

29. A method of determining proteomes of single cells comprising:
(a) synthesizing one or more gel chambers enclosing each of the one or more cells disposed on a surface of a channel;
(b) fixing and permeabilizing the one or more cells so that intracellular proteins of each cell are accessible to detection antibodies; and
(c) detecting the intracellular proteins with the detection antibodies.

30. The method of claim 29, further comprising collecting optical signals from the one or more cells disposed on the surface; and determining, prior to the synthesizing, the position of each of the one or more cells from the optical signals.
